SEQUENCE LISTING (8 sequences)

SEQ ID NO: 1 - DNA, 56 bp, synthetic construct
  source 1..56: mol_type = other DNA
cgggacctacattaagctggacaactcgagttgtccagcttaatgtaggtcttttt

SEQ ID NO: 2 - DNA, 58 bp, synthetic construct
  source 1..58: mol_type = other DNA
aattcaaaaagacctacattaagctggacaactcgagttgtccagcttaatgtaggtc

SEQ ID NO: 3 - DNA, 58 bp, synthetic construct
  source 1..58: mol_type = other DNA
ccggcgaggagttcaagacatacaactcgagttgtatgtcttgaactcctcgtttttg

SEQ ID NO: 4 - DNA, 58 bp, synthetic construct
  source 1..58: mol_type = other DNA
aattcaaaaacgaggagttcaagacatacaactcgagttgtatgtcttgaactcctcg

SEQ ID NO: 5 - DNA, 58 bp, synthetic construct
  source 1..58: mol_type = other DNA
ccgggctctcatcactccttcacttctcgagaagtgaaggagtgatgagagctttttg

SEQ ID NO: 6 - DNA, 58 bp, synthetic construct
  source 1..58: mol_type = other DNA
aattcaaaaagctctcatcactccttcacttctcgagaagtgaaggagtgatgagagc

SEQ ID NO: 7 - DNA, 58 bp, synthetic construct
  source 1..58: mol_type = other DNA
ccggcaacaagatgaagagcaccaactcgagttggtgctcttcatcttgttgtttttg

SEQ ID NO: 8 - DNA, 58 bp, synthetic construct
  source 1..58: mol_type = other DNA
aattcaaaaacaacaagatgaagagcaccaactcgagttggtgctcttcatcttgttg

(19) United States
(12) Patent Application Publication
(10) Pub. No.: US 2025/0179499 A1
(43) Pub. Date: Jun. 5, 2025

(54) USE OF CDK16 AS TARGET IN PREPARATION OF MEDICINE FOR TREATING TRIPLE-NEGATIVE BREAST CANCER

(71) Applicant: SHENZHEN TYERCAN BIO-PHARM CO., LTD., SHENZHEN (CN)

(72) Inventors: NATALIA VASILEVICH, SHENZHEN (CN); LICHUN SUN, SHENZHEN (CN); YUAN TIAN, SHENZHEN (CN); CHEGUO CAI, SHENZHEN (CN); MENGNA ZHANG, SHENZHEN (CN); XIAO LI, SHENZHEN (CN); LIYA YU, SHENZHEN (CN); LINGXIAN ZHANG, SHENZHEN (CN)

(73) Assignee: SHENZHEN TYERCAN BIO-PHARM CO., LTD., SHENZHEN (CN)

(21) Appl. No.: 18/844,950

(22) PCT Filed: Jan. 6, 2023
(86) PCT No.: PCT/CN2023/071088

§ 371 Date: Dec. 5, 2024

(30) Foreign Application Priority Data
Jan. 28, 2022 (CN) 202210104358.3

(51) Int. Cl. C12N 15/113; A61K 31/4709; A61P 35/00

(52) U.S. Cl. CPC C12N 15/1137; A61K 31/4709; A61P 35/00; C12N 2310/14; C12N 2310/531; C12Y 207/11022

(57) ABSTRACT

The present disclosure provides use of CDK16 as a target in preparation of a drug for treating triple-negative breast cancer. It is found through research in the present disclosure that knock down of the CDK16 can significantly inhibit the growth of TNBC xenograft tumors, PDX and PDO. Its mechanism is mainly that the knock down of the CDK16 can inhibit the phosphorylation of PRC1 (T481), prevent formation of spindles in a cell mitotic phase, and make cells unable to divide normally, thereby inhibiting the occurrence, development and metastasis of TNBC tumors. Meanwhile, it is found through research that Rebastinib, a covalent small-molecule inhibitor of the CDK16, shows a very good effect in inhibiting the development of TNBC tumors and is a potential novel drug for the treatment of TNBC.

### CROSS-REFERENCE OF RELATED APPLICATION

The present disclosure claims priority to a Chinese patent application No. CN202210104358.3 filed to the National Intellectual Property Administration on Jan. 28, 2022 and entitled “USE OF CDK16 AS TARGET IN PREPARATION OF DRUG FOR TREATING TRIPLE-NEGATIVE BREAST CANCER”, the entire content of which is incorporated by reference into the present disclosure.

### TECHNICAL FIELD

The present disclosure relates to the technical field of biomedicine, and in particular to use of CDK16 as a target in preparation of a drug for treating triple-negative breast cancer.

### BACKGROUND

Breast cancer has become a current malignant tumor with the highest incidence rate among women, posing a serious threat to women's life and health. Triple-negative breast cancer (TNBC) refers to a breast cancer subtype in which an estrogen receptor (ER), a progesterone receptor (PR) and a human epidermal growth factor receptor 2 (HER2) are all negative in a pathological staining report. It accounts for approximately 10%-15% of all breast cancers and is currently the most difficult subtype of breast cancer to treat. Currently, there is a lack of targeted drugs for the treatment of TNBC, and chemotherapy is still dominated. TNBC has a high mortality rate and is prone to recurrence and metastasis. Therefore, it is currently an urgent problem to be solved to find a new and effective treatment target for the TNBC.

Therefore, there is an urgent need to develop an effective TNBC therapeutic target and a drug for treating the triple-negative breast cancer.

### SUMMARY

The present disclosure provides use of CDK16 as a target in preparation of a drug for treating triple-negative breast cancer.

Optionally, the drug for treating the triple-negative breast cancer is a drug that inhibits the occurrence, growth and metastasis of the triple-negative breast cancer.

Optionally, the drug for treating the triple-negative breast cancer includes at least one of the following ingredients:
a CDK16 inhibitor; and
a reagent for knocking out CDK16.

Optionally, the reagent for knocking out CDK16 is a drug that inhibits the in vitro proliferation of triple-negative breast cancer cells, inhibits the growth of triple-negative breast cancer xenograft tumors, PDX and PDO; and the CDK16 inhibitor is a drug that inhibits the growth of triple-negative breast cancer tumors.

Optionally, the CDK16 inhibitor includes a small-molecule inhibitor Rebastinib.

Optionally, the reagent for knocking out CDK16 includes: a shRNA and/or gRNA that targets a target gene, wherein the shRNA nucleotide sequence is shown in SED ID NO. 1-SED ID NO. 2 or SED ID NO. 3-SED ID NO. 4 or SED ID NO. 5-SED ID NO. 6.

The present disclosure further provides use of the reagent for knocking out CDK16 or/and the CDK16 inhibitor in preparation of a drug for treating triple-negative breast cancer.

The present disclosure further provides a drug for treating triple-negative breast cancer, the drug including at least one of a CDK16 inhibitor and a reagent for knocking out CDK16.

The present disclosure further provides use of CDK16 as a target in a drug for treating triple-negative breast cancer.

The present disclosure further provides use of a reagent for knocking out CDK16 and/or a CDK16 inhibitor in a drug for treating triple-negative breast cancer.

The present disclosure further provides a method for treating triple-negative breast cancer, including:
administering the drug to a subject in need thereof.

Optionally, the reagent for knocking out CDK16 is a drug that inhibits the in vitro proliferation of triple-negative breast cancer cells, inhibits the growth of triple-negative breast cancer xenograft tumors, PDX and PDO; and the CDK16 inhibitor is a drug that inhibits the growth of triple-negative breast cancer tumors.

Optionally, the CDK16 inhibitor includes a small-molecule inhibitor Rebastinib.

Optionally, the reagent for knocking out CDK16 includes: a shRNA and/or gRNA targeting a target gene, wherein a nucleotide sequence of the shRNA is as shown in SED ID NO. 1-SED ID NO. 2 or SED ID NO. 3-SED ID NO. 4 or SED ID NO. 5-SED ID NO. 6.

### BRIEF DESCRIPTION OF THE DRAWINGS

In order to describe the technical solutions in the embodiments of the present disclosure more clearly, the following briefly describes the accompanying drawings required for describing the embodiments. Apparently, the accompanying drawings in the following description are only some embodiments of the present disclosure, and those of ordinary skills in the art may still derive other accompanying drawings from these accompanying drawings without creative efforts.

FIG. 1 shows the expression level and clinical relevance of CDK16 in different clinical databases of breast cancer, wherein: FIG. 1A shows the mRNA levels of CDK16 in different types of breast cancer in a TCGA database; FIG. 1B shows the correlation between CDK16 and an overall survival rate of patients; FIG. 1C shows the correlation between CDK16 and a progression-free survival rate of patients; FIG. 1D shows the correlation between CDK16 and the disease-free survival rate of patients; FIGS. 1E and 1F show the mRNA levels and overall survival rates of CDK16 in different types of breast cancer in a METABRIC database; FIG. 1G shows the mRNA expression levels of CDK16 in normal tissues and TNBC in GSE76250; and FIG. 1H shows the protein expression levels of CDK16 in normal tissues and breast cancer in a CPTAC database;

FIG. 2 shows the results of in vitro inhibition of TNBC cell line proliferation and cell apoptosis after CDK16 is knocked down, wherein: FIG. 2A shows that knock down of CDK16 significantly inhibits the in vitro proliferation of TNBC cell lines; and FIG. 2B shows that knock down of CDK16 significantly promotes the apoptosis of TNBC cell lines;

FIG. 3 shows the results that knock down of CDK16 significantly inhibits the growth of TNBC xenograft tumors, PDX and PDO, wherein: FIG. 3A shows that knock down of CDK16 significantly delays the occurrence of MDA-MB-231 tumors; FIG. 3B shows that knock down of CDK16 significantly reduces the volume of MDA-MB-231 tumors; FIGS. 3C and 3D show that knock down of CDK16 significantly reduces the weight and size of MDA-MB-231 tumors; FIG. 3E shows that knock down of CDK16 significantly reduces the size and number of TNBC-type PDOs; FIG. 3F shows that knock down of CDK16 significantly inhibits the proliferation of TNBC-type PDOs; FIG. 3G shows that knock down of CDK16 significantly delays the occurrence of TNBC-type PDX tumors; FIG. 3H shows that knock down of CDK16 significantly reduces the volumes of TNBC-type PDX tumors; and FIGS. 3I and 3J show that knock down of CDK16 significantly reduces the weights and sizes of TNBC-type PDX tumors;

FIG. 4 shows the result of knocking down CDK16 to inhibit TNBC metastasis and of overexpressing CDK16 to promote TNBC metastasis; wherein: FIG. 4A shows that knock down of CDK16 inhibits the systemic metastasis of 4T1 cells; and FIG. 4B shows that overexpression of CDK16 promotes the systemic metastasis of EMT6 cells;

FIG. 5 shows the result that a CDK16 small-molecule inhibitor Rebastinib significantly inhibits the growth of TNBC xenograft tumors, PDX and PDO; wherein: FIGS. 5A, 5B, 5C, and 5D show that Rebastinib significantly inhibits the volumes, weights, and sizes of MDA-MB-231 tumors; FIG. 5E shows that Rebastinib significantly inhibits the sizes of TNBC-type PDOs; FIG. 5F shows that Rebastinib significantly reduces the number of TNBC-type PDOs; FIG. 5G shows that Rebastinib significantly inhibits the proliferation of TNBC-type PDOs and promotes the apoptosis of TNBC-type PDOs; and FIGS. 5H, 5I, 5J, and 5K show that Rebastinib significantly inhibits the volumes, weights, and sizes of TNBC-type PDX tumors.

### DETAILED DESCRIPTION OF PREFERRED EMBODIMENTS

The present disclosure will be described in detail below in conjunction with specific embodiments and examples, and thus the advantages and various effects of the present disclosure will be presented more clearly. It should be understood by those of skills in the art that these specific embodiments and examples are used for illustrating the present disclosure, rather than limiting the present disclosure.

Throughout the specification, the terms used herein should be understood as having the meanings commonly used in the art, unless otherwise specifically stated. Therefore, all technical and scientific terms used herein have the same meaning as commonly understood by those of skills in the art to which the present disclosure belongs, unless defined otherwise. In case of conflict, the specification shall take precedence.

Unless otherwise specified, various raw materials, reagents, instruments, and devices, etc. used in the present disclosure can be commercially available or can be obtained through existing methods.

The technical solutions of the embodiments and examples of the present disclosure are to solve the aforementioned technical problems, and the overall idea is as follows:

CDK16 is knocked down in TNBC tumor cells (MDA-MB-231, PDX, PDO), and it is found by in vitro 3D culture and in vivo tumor formation experiments that knock down of CDK16 can significantly inhibit the occurrence and development of TNBC tumors.

In a model of TNBC tumor xenograft, PDO and PDX, it is found that the CDK16 small-molecule inhibitor Rebastinib can significantly inhibit the growth of TNBC tumors.

An embodiment of the present disclosure provides use of CDK16 as a target in preparation of a drug for treating triple-negative breast cancer.

In some embodiments, the drug for treating the triple-negative breast cancer is a drug that inhibits the occurrence, growth and metastasis of the triple-negative breast cancer.

In some embodiments, the drug for treating the triple-negative breast cancer includes at least one of the following ingredients:
a CDK16 inhibitor; and
a reagent for knocking out CDK16.

An embodiment of the present disclosure further provides use of the reagent for knocking out CDK16 or/and the CDK16 inhibitor in preparation of a drug for treating triple-negative breast cancer.

An embodiment of the present disclosure further provides a drug for treating triple-negative breast cancer, the drug including at least one of a CDK16 inhibitor and a reagent for knocking out CDK16.

In some embodiments, the drug further includes pharmaceutically-acceptable auxiliary materials and carriers. The auxiliary materials include at least one of a filler, a disintegrant, a binder, an excipient, a diluent, a lubricant, a sweetener or a colorant. The dosage form of the drug includes at least one of granules, tablets, pills, capsules, injections or dispersions.

An embodiment of the present disclosure further provides use of CDK16 as a target in a drug for treating triple-negative breast cancer.

An embodiment of the present disclosure further provides use of a reagent for knocking out CDK16 and/or a CDK16 inhibitor in a drug for treating triple-negative breast cancer.

An embodiment of the present disclosure further provides a method for treating triple-negative breast cancer, including:
administering the drug to a subject in need thereof.

In some embodiments, the reagent for knocking out CDK16 is a drug that inhibits the in vitro proliferation of triple-negative breast cancer cells, inhibits the growth of triple-negative breast cancer xenograft tumors, PDX and PDO; and the CDK16 inhibitor is a drug that inhibits the growth of triple-negative breast cancer tumors.

In some embodiments, the CDK16 inhibitor includes a small-molecule inhibitor Rebastinib.

In some embodiments, the reagent for knocking out CDK16 includes: a shRNA and/or gRNA targeting a target gene, wherein a nucleotide sequence of the shRNA is as shown in SED ID NO. 1-SED ID NO. 2 or SED ID NO. 3-SED ID NO. 4 or SED ID NO. 5-SED ID NO. 6.

It has been found through experiments in the present disclosure that targeting CDK16 (knock down) or the CDK16 inhibitor can inhibit the occurrence, development and metastasis of TNBC tumors in the model of TNBC tumor xenograft tumors, PDX and PDO.

The present disclosure provides use of CDK16 as a target in preparation of a drug for treating triple-negative breast cancer. It is found through research in the present disclosure that knock down of the CDK16 can significantly inhibit the growth of TNBC xenograft tumors, PDX and PDO. Its mechanism is mainly that the knock down of the CDK16 can inhibit the phosphorylation of PRC1 (T481), prevent formation of spindles in a cell mitotic phase, and make cells unable to divide normally, thereby inhibiting the occurrence, development and metastasis of TNBC tumors. Meanwhile, it is found through research that Rebastinib, a covalent small-molecule inhibitor of the CDK16, shows a very good effect in inhibiting the development of TNBC tumors and is a potential novel drug for the treatment of TNBC.

### EXAMPLES

The use of CDK16 as a target in preparation of a drug for treating triple-negative breast cancer in the present disclosure will be described in detail hereafter in conjunction with examples and experimental data.

Example 1 Knock down of CDK16 in TNBC tumor cells and investigation of tumor occurrence and development in a tumor cell line xenograft, PDX and PDO model

### 1. Vector Construction

(1) shRNA
1) Scramble shRNA (as a negative control group for interference):
positive-sense strand:

(SEDID NO. 7)
5′-CCGGCAACAAGATGAAGAGCACCAACTCGAGTTGGTGCTCTTCATC
TTGTTGTTTTTG-3′

anti-sense strand:

(SED ID NO. 8)
5′-AATTCAAAAACAACAAGATGAAGAGCACCAACTCGAGTTGGTGCTC
TTCATCTTGTTG-3′

2) CDK16-shRNA-1:
positive-sense strand:

(SEDID NO. 1)
5′-CGGGACCTACATTAAGCTGGACAACTCGAGTTGTCCAGCTTAATGT
AGGTCTTTTTG-3′

anti-sense strand:

(SED ID NO. 2)
5′-AATTCAAAAAGACCTACATTAAGCTGGACAACTCGAGTTGTCCAGC
TTAATGTAGGTC-3′

3) CDK16-shRNA-2:
positive-sense strand:

(SEDID NO. 3)
5′-CCGGCGAGGAGTTCAAGACATACAACTCGAGTTGTATGTCTTGAAC
TCCTCGTTTTTG-3′

anti-sense strand:

(SED ID NO. 4)
5′-AATTCAAAAACGAGGAGTTCAAGACATACAACTCGAGTTGTATGTC
TTGAACTCCTCG-3′

4) CDK16-shRNA-3:
positive-sense strand:

(SED ID NO. 5)
5′-CCGGGCTCTCATCACTCCTTCACTTCTCGAGAAGTGAAGGAGTGAT
GAGAGCTTTTTG-3′

anti-sense strand:

(SED ID NO. 6)
5′-AATTCAAAAAGCTCTCATCACTCCTTCACTTCTCGAGAAGTGAAGG
AGTGATGAGAGC-3′

(2) The positive-sense strand and anti-sense strand of each shRNA were annealed, and then a 58 bp oligonucleotide double strand was inserted into a pLKO.1 vector (the pLKO.1 vector is linearized by double digestion with EcoR1 and Age1 endonucleases, and then ligated enzymically) to obtain a CDK16-knockdown shRNA plasmid, wherein a GFP or mCherry gene sequence was contained on the pLKO.1 vector.

### 2. Lentiviral Packaging and Cell Transduction

The plasmid was extracted by using a DP118 kit (Tiangen Biotech). HEK293T cells were used for lentiviral packaging. The specific method was as follows.
(1) The HEK293T cells were plated in a 10 cm culture plate and cultured with a DMEM complete medium to ensure that the cells reached a confluence of 80%-90% when transfected the next day.
(2) Into a 950 μL Opti-MEM added was a total of 20 μg of a vesicular stomatitis virus G plasmid, a pCMV-48.9 packaging plasmid and a target plasmid at a mass ratio of 5:3:2, mixed well, then added with 60 μL of a PEI transfection reagent, and mixed well again by shaking, and allowed to stand for 15 min.
(3) Transfection of the HEK293T cells: The plasmids were evenly added dropwise into the HEK293T culture medium. After 6-8 h, the culture medium was removed by pipetting, then added with 10 mL of a fresh DMEM complete medium, and then cultured continually until 48 hours after transfection. The cell supernatant was collected. After centrifugation at 4,000 rpm for 10 min, the supernatant was filtered through a 0.45 μm filter membrane to obtain a lentiviral solution.
(4) Lentivirus infection of tumor cells: For infection of cell lines, cells were plated in a 6-well plate and cultured for 24 h. After adherence of the cells, the culture medium was removed by pipetting and replaced with a lentiviral solution, and replaced with a fresh culture medium after 24 h and continually cultured for another 24 h. Cell fluorescence was observed under a fluorescence microscope, and positive cells were sorted out by a flow cytometer to establish a line and amplify it for subsequent experiments. For infection of a patient with primary cells, primary tumor single cells were isolated from a tissue of the patient. The cells were plated in a 6-well plate or culture dish, added with a lentiviral solution, and suspended and infected for 24 h. Then the cells were collected by centrifugation. Positive cells were sorted by a flow cytometer for the construction of PDX or PDO.
   3. In-Situ Transplantation of TNBC Tumor Cell Lines into Tumors

Lentivirus-infected breast tumor cells were resuspended with 50% of FBS (diluted with PBS), added with Matrigel at a volume ratio of 1:1, then added with 0.04% of trypan blue, mixed well, and placed on ice until inoculation into mice. After the mouse was anesthetized, it was placed on an experimental table with its abdomen facing upwards, and a “Y”-shaped opening was cut in its abdominal epidermis along a midline and the direction of the lower limbs on both sides with scissors (without cutting the peritoneum). The opening was peeled off to both sides with cotton swabs and tweezers to expose the fourth pair of mammary gland fat pads, and the epidermis was fixed with needles. 10-20 μL of a cell suspension was drawn up with a syringe and injected into the mammary gland fat pads. Finally, the skin on both sides was closed and sutured with surgical staples. The surgical staples could be removed after the wound healed. Since the surgery, the occurrence of tumors in the mouse was observed and recorded every day. After about one week, the length and width of the tumor were measured with a vernier caliper and recorded, and the change in the weight of the mouse was monitored. The tumor volume was calculated by the following formula: Volume (mm³)=length×width×width×0.52.

### 4. Construction of PDX Model

Primary tumor cells from the patient infected with the lentivirus were in-situ injected into the fourth pair of mammary gland fat pads of the NSG mice according to the method described in 3, and the occurrence of tumors was observed and recorded.

### 5. Establishment of PDO Model

The primary tumor cells from the patient infected with the lentivirus were resuspended with pre-cooled Matrigel to a concentration of 2× 10⁴ cells/50 μL Matrigel. The Matrigel was added dropwise into a 24-well culture plate and allowed to stand at 37° C. for 15-20 min. After confluence, the cells were added with a breast cancer tumor organoid culture medium for culture, the culture medium was replaced by a fresh culture medium every three days, and the number and size of organoid growth were counted.

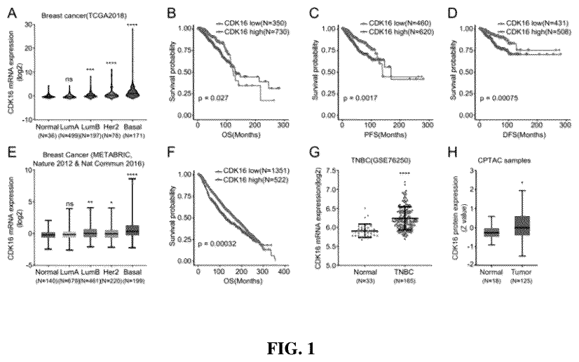

The results were as described in FIGS. 1-4.

It could be seen from FIG. 1 that, CDK16 was highly expressed in breast cancer, especially triple-negative breast cancer, and was correlated with a clinical survival rate.

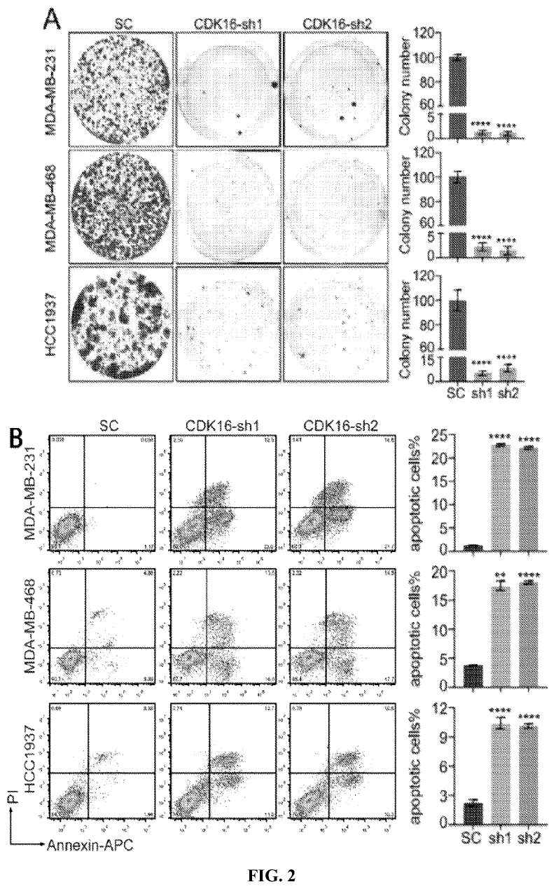

It could be seen from FIG. 2 that, knock down of CDK16 inhibited the proliferation of TNBC cell lines in vitro and led to cell apoptosis.

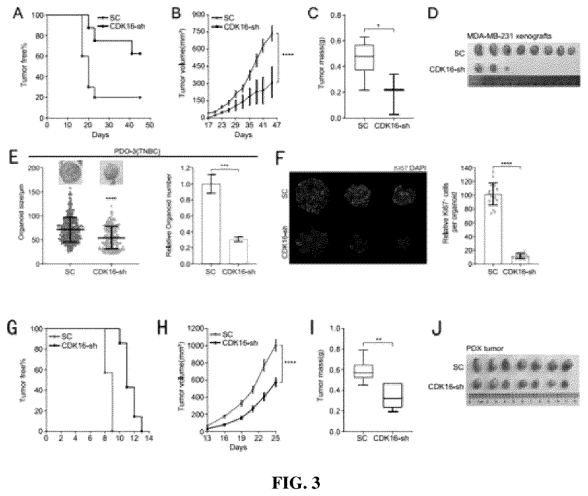

It could be seen from FIG. 3 that, knock down of CDK16 significantly inhibited the growth of TNBC xenograft tumors, PDX, and PDO.

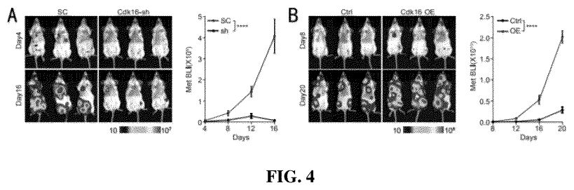

It could be seen from FIG. 4 that, knock down of CDK16 inhibited TNBC metastasis, while overexpression of CDK16 promoted TNBC metastasis.

### Example 2 Investigation of the Inhibitory Effect of CDK16 Inhibitor Rebastinib on Tumor Growth and Metastasis

### 1. Inhibition of the Growth of MDA-MB-231 Xenografts and PDX by Rebastinib

The tumor cell line was transplanted in situ as that in Example 1. When the tumor size reached 50 cm³, the mice were divided into a control group (solvent) and a group administrated with Rebastinib (80 mg/kg), with 3 mice in each group. The mice were administrated by gavage continuously (administrated with MDA-MB-231 xenografts for 13 days and administrated with PDX for 11 days), during which the mice were weighed every day, and the tumor size was measured every three days. After completion of the administration, the mice were sacrificed and the tumors were weighed.

### 2. Inhibition of the Growth of TNB PDO by Rebastinib

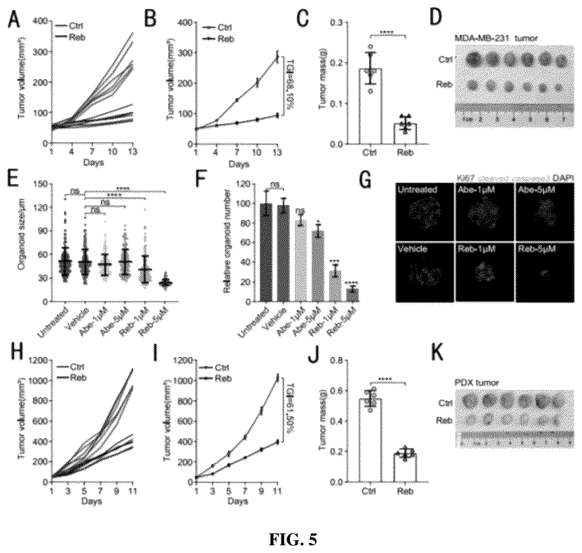

Similar to the PDO model establishment operation in Example 1, the primary tumor cells derived from TNBC patients were resuspended with pre-cooled Matrigel to a concentration of 2×10⁴ cells/50 μL Matrigel. The Matrigel was added dropwise into a 24-well culture plate and allowed to stand at 37° C. for 15-20 min. After confluence, the cells were added with a breast cancer tumor organoid culture medium for culture, and the culture medium was replaced by a fresh culture medium every three days. The PDOs were divided into 6 groups, including an untreated group, a solvent control group, groups administrated with low and high doses of Abemaciclib (with doses of 1 μM and 5 μM, respectively), and groups administrated with low and high doses of Rebastinib (with doses of 1 μM and 5 μM, respectively). After three days of culture, the PDOs were started to be treated by addition of a drug according to groups. The size and number of organoids were counted after one week. The results were as shown in FIG. 5.

As shown in FIG. 5, the CDK16 small-molecule inhibitor Rebastinib significantly inhibited the growth of TNBC xenograft tumors, PDX and PDO.

Finally, it should be noted that the terms “include”, “comprise” or any other variations thereof are intended to cover non-exclusive inclusion, so that a process, method, substance or device including a series of elements includes not only those elements, but also other elements not explicitly listed, or the elements inherent to such process, method, substance or device.

Although alternative embodiments of the present disclosure have been described, additional changes and modifications may be made to these embodiments once those skilled in the art are aware of the basic inventive concepts. Therefore, it is intended that the appended claims are interpreted to include alternative embodiments and all changes and modifications that fall within the scope of the present disclosure.

Obviously, various changes and modifications can be made by those skilled in the art to the present disclosure without departing from the spirit and scope of the present disclosure. As such, if these modifications and variations of the present disclosure fall within the scope of the claims of the present disclosure and their equivalent technologies, the present disclosure is also intended to include these modifications and variations.

### INDUSTRIAL APPLICABILITY

It is found through research in the present disclosure that knock down of the CDK16 can significantly inhibit the growth of TNBC xenograft tumors, PDX and PDO. Its mechanism is mainly that the knock down of the CDK16 can inhibit the phosphorylation of PRC1 (T481), prevent formation of spindles in a cell mitotic phase, and make cells unable to divide normally, thereby inhibiting the occurrence, development and metastasis of TNBC tumors. Meanwhile, it is found through research that Rebastinib, a covalent small-molecule inhibitor of the CDK16, shows a very good effect in inhibiting the development of TNBC tumors and is a potential novel drug for the treatment of TNBC. Therefore, it has excellent practicality.

1. Use of CDK16 as a target in preparation of a drug for treating triple-negative breast cancer.

2. The use according to claim 1, wherein the drug for treating the triple-negative breast cancer is a drug that inhibits the occurrence, growth and metastasis of the triple-negative breast cancer.

3. The use according to claim 2, wherein the drug for treating triple-negative breast cancer comprises at least one of the following ingredients:
a CDK16 inhibitor; and a reagent for knocking out CDK16.

4. The use according to claim 3, wherein the reagent for knocking out CDK16 is a drug that inhibits the in vitro proliferation of triple-negative breast cancer cells, inhibits the growth of triple-negative breast cancer xenograft tumors, PDX and PDO; and the CDK16 inhibitor is a drug that inhibits the growth of triple-negative breast cancer tumors.

5. The use according to claim 3, wherein the CDK16 inhibitor comprises a small-molecule inhibitor Rebastinib.

6. The use according to claim 3, wherein the reagent for knocking out CDK16 comprises: a shRNA and/or gRNA targeting a target gene, wherein a nucleotide sequence of the shRNA is as shown in SED ID NO. 1-SED ID NO. 2 or SED ID NO. 3-SED ID NO. 4 or SED ID NO. 5-SED ID NO. 6.

7. Use of the reagent for knocking out CDK16 or/and the CDK16 inhibitor in preparation of a drug for treating triple-negative breast cancer.

8. The use according to claim 7, wherein a drug for treating triple-negative breast cancer comprises at least one of a CDK16 inhibitor and a reagent for knocking out CDK16.

9. Use of CDK16 as a target in a drug for treating triple-negative breast cancer.

10. Use of a reagent for knocking out CDK16 and/or a CDK16 inhibitor in a drug for treating triple-negative breast cancer.

11. A method of treating triple-negative breast cancer, comprising:
administering the drug according to claim 8 to a subject in need thereof.

12. The use according to claim 10, wherein the reagent for knocking out CDK16 is a drug that inhibits the in vitro proliferation of triple-negative breast cancer cells, inhibits the growth of triple-negative breast cancer xenograft tumors, PDX and PDO; and the CDK16 inhibitor is a drug that inhibits the growth of triple-negative breast cancer tumors.

13. The use according to claim 10, wherein the CDK16 inhibitor comprises a small-molecule inhibitor Rebastinib.

14. The use according to claim 10, wherein the reagent for knocking out CDK16 comprises: a shRNA and/or gRNA targeting a target gene, wherein a nucleotide sequence of the shRNA is as shown in SED ID NO. 1-SED ID NO. 2 or SED ID NO. 3-SED ID NO. 4 or SED ID NO. 5-SED ID NO. 6.
